(19) United States
(12) Patent Application Publication
(10) Pub. No.: US 2004/0191294 A1
(43) Pub. Date: Sep. 30, 2004

(54) Health composition

(76) Inventors: Talahalli Ravichandra Ramaprasad, Mysore (IN); Vallikannan Bhaskaran, Mysore (IN); Sukumar Debnath, Mysore (IN); Kari Sambaiah, Mysore (IN); Samkaramthadathil Gangadharan Jayaprakashan, Mysore (IN); Mahadevaiah Shivakumar, Mysore (IN); Belur R. Lokesh, Mysore (IN)

Correspondence Address: SHLESINGER, ARKWRIGHT & GARVEY LLP, 3000 SOUTH EADS STREET, ARLINGTON, VA, 22202, US

(21) Appl. No.: 10/397,843

(22) Filed: Mar. 27, 2003

(51) Int. Cl. A61K 47/00; A61K 31/202

(52) U.S. Cl. 424/439; 514/560

(57) ABSTRACT

A health composition comprising milk or milk-based products and a desired amount of one or more omega 3 fatty acids, a method for preparing the composition and a method for reducing cholesterol and triglyceride levels in a subject by administering the said composition.

### FIELD OF THE INVENTION

The present invention relates to a health composition, a method for preparing the composition and a method for reducing cholesterol and triglyceride levels in a subject by administering the said composition. More particularly, the present invention relates to a synergistic milk based composition enriched with omega 3 fatty acids obtained from natural sources for lowering cholesterol and triglyceride levels in mammals.

### BACKGROUND AND PRIOR ART DESCRIPTION

Milk is an important fluid that has evolved to sustain life for the new born mammals. Milk contains wider range of nutrients than most other foods. Apart from contributing a wide range of nutrients, milk also supplies a high proportion of calcium and riboflavin. Milk, has high nutrient density; as they provide high intakes of nutrients per unit of food energy. For most of these nutrients the bio availability is generally high compared with those of vegetable foods, and milk may also enhance the bioavailability of nutrients from other foods. Milk is a flexible food and its products differ widely in water, fats, lactose, protein, vitamin and mineral contents, contributing to a wide variety of eating patterns and fulfilling many nutritional uses and needs. In addition to these traditional roles, milk is now regarded as able to provide a certain protection against ill health and positively to promote good health. Thus, some constituents of milk play a role in protecting against dental decay: others have anti cancer properties; yet other reduce blood pressure and have anti thrombotic effects. Several constituents of milk are important in bone health. Digestion and/or fermentation of milk proteins give rise to peptides that improve immune function and influence the nervous system. Milk is a good source of medium chain fatty acids which can be easily absorbed by either portal or lymphatic systems. Medium chain fatty acids offer numerous health benefits. They are rapidly metabolized to yield quick energy and are not deposited in the body as fat. Medium chain triglycerides have clinical application in the treatment of fat malabsorption disorders, gall bladder disease, hyperlipidemia, obesity and in deficiency of carnitine system. Milk contains a number of compounds such as conjugated linoleic acid, sphingomylein, butyric acid, ether lipids, β carotene and vitamins A and B that have anti cancer potential. Sphingomylein, through its metabolites ceramide and sphingosine, participates in multiple antiproliferative pathways associated with suppression of carcinogenesis. Butyric acid uniquely present in milk is a potent antineoplastic agent. Dietary butyric acid inhibits mammary carcinoma development in rats. In humans, ether lipids, β carotene and vitamins A and D are associated with anticancer effects.

Thus milk is uniquely enriched in many beneficial components and widely used by people of different age groups. However, it is devoid of fatty acids of omega 3 family like α linolenic acid (LNA), eicosapentaenoic acid (EPA) docosahexaenoic acid (DHA). These fatty acids have several health benefits including controlling cardiovascular diseases, immune disorders and inflammation. These fatty acids are also essential for the development of the brain and retina in humans. Studies conducted on non human primates and also humans suggest that DHA is essential for the normal functioning of the retina and brain. Studies have also shown that omega 3 fatty acids can decrease number and size of tumors and increase the time elapsed before the appearance of tumors.

Metabolically, EPA is an antagonist of the arachidonic acid cascade and competes with arachidonic acid for cyclooxygenase and lipooxygenase to produce eicosanoids. EPA is used for the synthesis of eicosanoids such as series 3 prostaglandins and ameliorates immune dysfunction. High levels of omega 3 fatty acids in the diet lead to substitution of some arachidonic acid by EPA and DHA. The change in the eicosanoid synthesis seen with omega 3 fatty acids feeding is associated with an improved immune competence and a reduced inflammatory response to injury.

Milk is an ideal vehicle for delivering these beneficial fatty acids as it is consumed by large section of population in this country.

In the present work linseed oil, nature's richest source of α-linolenic acid is used as omega 3 fatty acids source to supplement milk with α-linolenic acid. Linseed oil contains 55% of total fatty acids as α-linolenic acid. Linseed oil obtained from linseed, one of the worlds most ancient cultivated plants, α-linolenic acid and other omega 3 fatty acids offers significant benefit in many diseases linked to modern living, including the three major killers heart disease, cancer and strokes. Some conditions improved by supplementing the diet with omega 3 oils include lowering of serum cholesterol levels, prevention of strokes and heart attacks, angina, high blood pressure, rheumatoid arthritis, multiple sclerosis, psoriasis, eczema, cancer prevention and treatments.

In the present work, fish oil, yet another source of long chain omega 3 fatty acids like EPA and DHA is used to supplement milk with omega 3 fatty acids. Studies have shown supplementation of diets with fish oils demonstrates many benefits, especially in improving cardiovascular function including lowering of cholesterol levels and blood pressure.

The methodology described here involves the production of cholesterol lowering health beverage mix supplemented with linseed oil or fish oil as a source of omega 3 fatty acids.

Reference may be made to the method for producing milk product having high concentration of omega 3 highly unsaturated fatty acids (U.S. Pat. No. 6,177,108) where in, a process for the aerobic culture if fungi of the order Thraustochytriales for the production of whole celled or extracted microbial products with a high concentration of omega 3 fatty acids is described. The harvested whole-cell microbial product can be added to processed foods as nutritional supplement and also can be added to cattle feeds to enhance the omega 3 fatty acids content of the milk.

The disadvantage of the product of the above processes is the acceptability of microbial extract or the whole celled product containing omega 3 fatty acids for food supplementation.

Another reference may be made in which dairy products with enhanced conjugated linoleic acid (CLA) and omega 3 fatty acid is discussed (Pat. No. WO 9908540). The product enriched with CLA and other beneficial unsaturated fatty acid obtained by feeding cattle with diet which includes a fish-derived product such as fish oil or fish meal.

Yet another reference may be made to a patent (EP 1106073), in which poly unsaturated fatty acids enriched milk is produced by feeding cows with diet containing extruded linseed granules.

In the above two references the diet rich in omega 3 fatty acids are fed to cattle to produce omega 3 fatty acid containing milk. This approach will be not very advantageous as there may be metabolic variations due to the seasons that can alter the final fatty acid composition of the milk.

Yet another reference may be made to patent (U.S. Pat. No. 5,985.348) disclosing a methodology for the microbial fermentation to produce microbial product which can be added to processed foods or can be used as a supplement in cattle feed to produce milk containing omega 3 fatty acids.

It may be noted that the reference mentioned above involves processes that are often complicated, as it involves extensive protocol for cell culture, maintenance and formulation of feed with cultured cells as a source of PUFA.

Where as the present invention involves the direct supplementation of omega 3 fatty acids from natural sources to the milk by physical blending using homogenizers and spray dying to get a final product in the powder form enriched with omega 3 fatty acids.

The present invention also provides a scope for the enrichment of milk with omega 3 fatty acids at desired levels by altering the amount of omega 3 fatty acid source added to the milk.

### OBJECTS OF THE PRESENT INVENTION

The main objective of the present invention is to provide a health composition capable of reducing cholesterol and triglyceride levels in a subject

Another object of the present invention is to provide a synergistic milk based health composition enriched in omega 3 fatty acids capable of reducing cholesterol and triglyceride levels in mammals.

Yet another object of the present invention is to provide a process for preparing the milk based health composition enriched with omega 3 fatty acids from natural sources.

Still another objective of the present invention is to provide a process for preparing a free flowing, spray dried and reconstitutable milk based health composition enriched with α-linolenic acid or EPA and DHA obtained from natural sources at predetermined levels.

A further object of the present invention is to provide a method for reducing cholesterol and triglyceride levels in a mammal using the health composition.

### SUMMARY OF THE INVENTION

The present invention provides a health composition comprising milk or milk-based products and a desired amount of one or more omega 3 fatty acids, a method for preparing the composition and a method for reducing cholesterol and triglyceride levels in a subject by administering the said composition.

### DETAILED DESCRIPTION OF THE PRESENT INVENTION

Accordingly, the present invention provides a synergistic health composition comprising milk or milk-based products and a desired amount of one or more omega 3 fatty acids.

In an embodiment of the present invention, the ratio between omega 3 fatty acids and milk or milk-based product is in the range of 1:100 to 1:25.

In another embodiment of the present invention, the ratio between omega 3 fatty acids and milk or milk-based product is 1:50.

In yet another embodiment of the present invention, the omega 3 fatty acids are obtained from natural source.

In still another embodiment of the present invention, the omega 3 fatty acids are seleted from α linolenic acid (LNA), eicosapentaenoic acid (EPA) and docosahexaenoic acid (DHA).

In one more embodiment of the present invention, α linolenic acid is obtained from linseed oil or fish oil.

In one another embodiment of the present invention, EPA and DHA are obtained from fish oil.

In a further embodiment of the present invention, the milk based product includes any food grade additions such as proteins, sugars, fats, fibres, colouring agents, preservatives, herbal material, pharmaceuticals, mineral mix and vitamin mix.

In another embodiment of the present invention, the milk-based product contains:

| Ingredients | Quantity (g) |
|---|---|
| Maltodextrin | 3000 to 3500 |
| Potassium citrate | 50 to 55 |
| Sodium citrate | 50 to 55 |
| K₂HPO₄ | 75 to 100 |
| Skimmed milk powder | 700 to 1000 |
| Calcium gluconate | 300 to 400 |
| Vitamin mix | 25 to 40 |
| Mineral mix | 5 to 10 |
| Sugar | 2000 to 5000 |

for every 50 liters of milk.

In yet another embodiment of the present invention, the milk-based product contains:

| Ingredients | Quantity (g) |
|---|---|
| Maltodextrin | 3050.0 |
| Potassium citrate | 52.5 |
| Sodium citrate | 52.86 |
| K₂HPO₄ | 80.34 |
| Skimmed milk powder | 720.0 |
| Calcium gluconate | 357.14 |
| Vitamin mix | 30.0 |
| Mineral mix | 8.12 |
| Sugar | 2320.0 |

for every 50 litres of milk.

In still another embodiment of the present invention, the vitamin mix includes vitamin A, vitamin D, vitamin K, thiamine, riboflavin, niacin, vitamin B6, folic acid, biotin, pantothenic acid, choline bitartrate, inositol and vitamin C.

In one more embodiment of the present invention, the milk based product includes multiple Vitamins such as in the range of 200,000 IU vitamin A, 72,000 IU vitamin D, 0.80 g vitamin D, 2.57 mg vitamin K, 51.4 mg thiamine, 128.57 mg riboflavin, 0.771 g niacin, 64.28 mg vitamin B6, 6.42 mg folic acid, 3.57 mg biotin, 257.14 mg pantothenic acid, 2.57 g choline bitartrate, 0.84 g inositol, 7.71 g vitamin C.

In one another embodiment of the present invention, the mineral mix includes FeSO₄, ZnSO₄, CuSO₄, KI and MnSO₄.

In a further embodiment of the present invention, the milk-based product includes 6.42 g FeSO₄ (IP grade), 1.71 g ZnSO₄, 0.12 g CuSO₄, 4.2 mg KI, 10.71 MnSO₄.

In an embodiment of the present invention, the cholesterol lowering capacity of the composition is in the range of 20-30%.

In another embodiment of the present invention, the composition lowers the cholesterol levels in serum and liver.

In yet another embodiment of the present invention, the triglyceride lowering capacity of the composition is in the range of 20 to 35%.

In still another embodiment of the present invention, the composition lowers the triglyceride levels in serum and liver

The present invention also provides a process for preparing a synergistic cholesterol lowering health composition, said process comprising mixing milk or milk based products with a desired quantum of omega 3 fatty acids.

In an embodiment of the present invention, omega 3 fatty acids and milk or milk-based product are mixed in a ratio ranging between 1:100 to 1:25.

In another embodiment of the present invention, omega 3 fatty acids and milk or milk-based product are mixed in the ratio of 1:50.

In yet another embodiment of the present invention, the omega 3 fatty acids are obtained from natural sources.

In still another embodiment of the present invention, the omega 3 fatty acids are selected from α linolenic acid (LNA), eicosapentaenoic acid (EPA) and docosahexaenoic acid (DHA).

In one more embodiment of the present invention, α linolenic acid is obtained from linseed oil or fish oil.

In one another embodiment of the present invention, EPA and DHA are obtained from fish oil.

In a further embodiment of the present invention, the milk-based product includes food grade additions such as proteins, sugars, fats, fibres, colouring agents, preservatives, herbal material, pharmaceuticals, mineral mix and vitamin mix.

In an embodiment of the present invention, the milk-based product contains:.

| Ingredients | Quantity (g) |
|---|---|
| Maltodextrin | 3000 to 3500 |
| Potassium citrate | 50 to 55 |
| Sodium citrate | 50 to 55 |
| K₂HPO₄ | 75 to 100 |
| Skimmed milk powder | 700 to 1000 |
| Calcium gluconate | 300 to 400 |
| Vitamin mix | 25 to 40 |
| Mineral mix | 5 to 10 |
| Sugar | 2000 to 5000 |

for every 50 liters of milk.

In another embodiment of the present invention, the milk-based product contains:

| Ingredients | Quantity (g) |
|---|---|
| Maltodextrin | 3050.0 |
| Potassium citrate | 52.5 |
| Sodium citrate | 52.86 |
| K₂HPO₄ | 80.34 |
| Skimmed milk powder | 720.0 |
| Calcium gluconate | 357.14 |
| Vitamin mix | 30.0 |
| Mineral mix | 8.12 |
| Sugar | 2320.0 |

for every 50 litres of milk.

In yet another embodiment of the present invention, the vitamin mix includes vitamin A, vitamin D, vitamin K, thiamine, riboflavin, niacin, vitamin B6, folic acid, biotin, pantothenic acid, choline bitartrate, inositol and vitamin C.

In still another embodiment of the present invention, the milk based product includes multiple Vitamins such as in the range of 200,000 IU vitamin A, 72,000 IU vitamin D, 0.80 g vitamin D, 2.57 mg vitamin K, 51.4 mg thiamine, 128.57 mg riboflavin, 0.771 g niacin, 64.29 mg vitamin B6, 6.42 mg folic acid, 3.57 mg biotin, 257.14 mg pantothenic acid, 2.57 g choline bitartrate, 0.84 g inositol, 7.71 g vitamin C.

In one more embodiment of the present invention, the mineral mix includes FeSO₄, ZnSO₄, CuSO₄, KI and MnSO₄.

In one another embodiment of the present invention, the milk-based product includes 6.42 g FeSO₄ (IP grade), 1.71 g ZnSO₄, 0.12 g CuSO₄, 4.2 mg KI, 10.71 MnSO₄.

More particularly, the present invention provides a process for preparing the health composition, said process comprises the steps of:

(a) physically blending milk or milk-based product with omega 3 fatty acids obtained from natural sources to obtain a milk or milk-based product enriched with omega 3 fatty acids;

(b) homogenizing the milk or milk-based product enriched with omega 3 fatty acids of step (a);

(c) partially condensing the homogenized milk or milk-based product of step (b) under vacuum to obtain desired brix, and

(d) spray drying the partially condensed milk or milk based product of step (c) to form powder.

In an embodiment of the present invention, the cholesterol lowering capacity of the composition is in the range of 20-30%.

In another embodiment of the present invention, the composition lowers the cholesterol level in serum and liver.

In yet another embodiment of the present invention, the triglyceride lowering capacity of the composition is in the range of 20 to 35%.

In still another embodiment of the present invention, the composition lowers the triglyceride levels in serum and liver.

The present invention further provides a method for reducing total cholesterol and triglycerides in a subject, said method comprising the step of administering an effective amount of a composition comprising milk or milk-based products and a desired amount of one or more omega 3 fatty acids.

In an embodiment of the present invention, the ratio between omega 3 fatty acids and milk or milk-based product is in the range of 1:100 to 1:25.

In another embodiment of the present invention, the ratio between omega 3 fatty acids and milk or milk-based product is 1:50.

In yet another embodiment of the present invention, the omega 3 fatty acids are obtained from natural source.

In still another embodiment of the present invention, the omega 3 fatty acids are seleted from α linolenic acid (LNA), eicosapentaenoic acid (EPA) and docosahexaenoic acid (DHA).

In one more embodiment of the present invention, α linolenic acid is obtained from linseed oil or fish oil.

In one another embodiment of the present invention, EPA and DHA are obtained from fish oil.

In a further embodiment of the present invention, the milk based product includes any food grade additions such as proteins, sugars, fats, fibres, colouring agents, preservatives, herbal material, pharmaceuticals, mineral mix and vitamin mix.

In an embodiment of the present invention, the milk-based product contains:

| Ingredients | Quantity (g) |
|---|---|
| Maltodextrin | 3000 to 3500 |
| Potassium citrate | 50 to 55 |
| Sodium citrate | 50 to 55 |
| K₂HPO₄ | 75 to 100 |
| Skimmed milk powder | 700 to 1000 |
| Calcium gluconate | 300 to 400 |
| Vitamin mix | 25 to 40 |
| Mineral mix | 5 to 10 |
| Sugar | 2000 to 5000 |

for every 50 liters of milk.

In another embodiment of the present invention, the milk-based product contains:

| Ingredients | Quantity (g) |
|---|---|
| Maltodextrin | 3050.0 |
| Potassium citrate | 52.5 |
| Sodium citrate | 52.86 |
| K₂HPO₄ | 80.34 |
| Skimmed milk powder | 720.0 |
| Calcium gluconate | 357.14 |
| Vitamin mix | 30.0 |
| Mineral mix | 8.12 |
| Sugar | 2320.0 |

for every 50 litres of milk.

In yet another embodiment of the present invention, the vitamin mix includes vitamin A, vitamin D, vitamin K, thiamine, riboflavin, niacin, vitamin B6, folic acid, biotin, pantothenic acid, choline bitartrate, inositol and vitamin C.

In still another embodiment of the present invention, the milk based product includes multiple Vitamins such as in the range of 200,000 IU vitamin A, 72,000 IU vitamin D, 0.80 g vitamin D, 2.37 mg vitamin K, 51.4 mg thiamine, 128.57 mg riboflavin, 0.771 g niacin, 64.28 mg vitamin B6, 6.42 mg folic acid, 3.57 mg biotin, 257.14 mg pantothenic acid, 2.57 g choline bitartrate, 0.84 g inositol, 7.71 g vitamin C.

In one more embodiment of the present invention, the mineral mix includes FeSO₄, ZnSO₄, CuSO₄, KI and MnSO₄.

In one another embodiment of the present invention, the milk-based product includes 6.42 g FeSO₄ (IP grade), 1.71 g ZnSO₄, 0.12 g CuSO₄, 4.2 mg KI, 10.71 MnSO₄.

In an embodiment of the present invention, the composition is administered at 10 to 20% level of far to the subject.

In another embodiment of the present invention, the composition is administered for a period of 45 to 90 days.

In yet another embodiment of the present invention the subject is a mammal including human being.

In still another embodiment of the present invention, the composition lowers the cholesterol content in the range of 20-30%.

In one more embodiment of the present invention, the composition lowers the cholesterol level in serum and liver.

In one another embodiment of the present invention, the composition lowers the triglycerides content by 20 to 35%.

In a further embodiment of the present invention, the composition lowers the triglyceride levels in serum and liver

Accordingly the present invention provides a methodology for the production of cholesterol lowering milk based product supplemented with omega 3 fatty acids from natural sources (linseed oil or fish oil) which comprises as follows.

1 Supplementation of omega 3 fatty acids to milk by physical mixing of omega 3 fatty acid source at a specific amount.

2. Omega 3 fatty acid supplemented milk will be provided with necessary amount of vitamins and minerals.

3. Spray drying the supplemented milk at suitable temperature results in the milk based product in powder form enriched with omega 3 fatty acids.

4. The product will be packed under nitrogen.

5. The product has the cholesterol lowering capacity in the range of 20-35%.

The process flow chart is shown below.

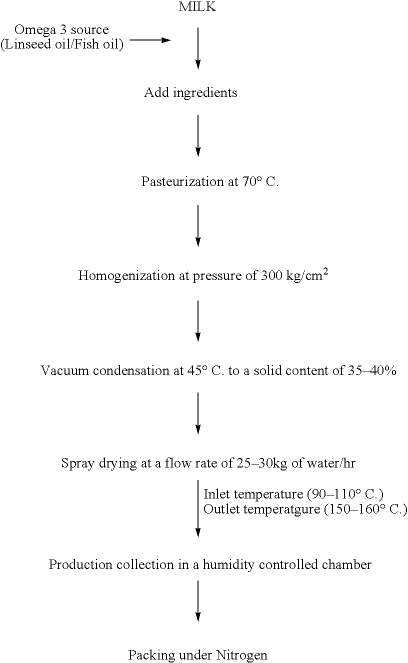

The novelty of the present invention is the development of a process for the production of milk based product supplemented with useful fatty acids from natural sources.

The novelty of the product of the process is that it contains the medium chain fatty acids of milk and omega 3 polyunsaturated fatty acids from natural sources, which is nutritionally advantageous by way of being hypocholesterolemic and hypotriglyceridemic.

The following examples are given by way of illustrations of the present invention and therefore should not be constructed to limit the scope of the present invention.

### EXAMPLE 1

To 50 liters of milk, vitamins, minerals, calcium gluconate, skimmed milk powder and sugar is added. The composition of ingredients added to 50 litre of milk during processing is given in table 1. An omega 3 fatty acid source (linseed oil or fish oil) is added at 2% level. Homogenization is carried out using two stage homogenizer at 300 kg/cm² and condensed under vacuum to a solid content of 35 to 40%. Another formulation containing GNO at the same level as LSO or FO was also prepared. Milk powder is obtained by spray drying. The inlet temperature was 90-110° C. and the outlet temperature was 150-160° C., flow rate was kept at 25-30 liters/hr. The powder is collected in a humidity controlled chamber and packed under nitrogen.

TABLE 1
Composition of ingredients added to 50 liter of milk during processing

| Ingredients | Quantity (g) |
|---|---|
| Maltodextrin | 3050.0 |
| Potassium citrate | 52.5 |
| Sodium citrate | 52.86 |
| K₂HPO₄ | 80.34 |
| Skimmed milk powder | 720.0 |
| Calcium gluconate | 357.14 |
| Vitamin mix* | 30.0 |
| Mineral mix** | 8.12 |
| Sugar | 2320.0 |
| Oil | 1000.0 |

*Vitamin mix added includes; 200,000 IU vitamin A, 72,000 IU vitamin D, 0.80 g vitamin D, 2.57 mg vitamin K, 51.4 mg thiamine, 128.57 mg riboflavin, 0.771 g niacin. 64.28 mg vitamin B₆, 6.42 mg folic acid, 3.57 mg biotin, 257.14 mg pantothenic acid, 2.57 g choline bitartrate, 0.84 g inositol, 7.71 g vitamin C.
**Mineral mix added includes; 6.42 g FeSO₄ (IP grade), 1.71 g ZnSO₄ 0.12 g CuSO₄, 4.2 mg KI, 10.71 MnSO₄.
Yield of the dry mix: 10.0 kg

The fatty acid composition of milk, Ground nut oil (GNO), Linseed oil (LSO), and Fish oil (FO) used in the present invention is given in table 2.

TABLE 2
Fatty acid composition of fats used in the preparation of milk powder

| | | Ground nut oil | | |
|---|---|---|---|---|
| Fatty acids mol % | Milk | (control) | Linseed oil | Fish oil |
| Caprylic acid | 0.63 | — | — | — |
| Lauric acid | 1.84 | — | — | — |
| Myristic acid | 9.68 | 1.7 | — | 5.8 |
| Palmitic acid | 33.0 | 17.5 | 6.04 | 15.1 |
| Palmitoleic acid | 1.7 | — | — | 11.7 |
| Stearic acid | 9.8 | 2.0 | 4.5 | 4.41 |
| Oleic acid | 37.2 | 37.9 | 25.2 | 24.7 |
| Linoleic acid | 6.1 | 36.0 | 12.01 | 5.15 |
| Linolenic acid | — | — | 52.25 | 1.6 |
| Arachidic acid | — | 2.8 | — | 8.24 |
| Arachidonic acid | — | — | — | 7.4 |
| Eicosapentaenoic acid | — | — | — | 6.64 |
| Behenic acid | — | 2.0 | — | — |
| Docosahexaenoic acid | — | — | — | 9.24 |

The analysis of fatty acid by gas chromatography showed an increase in α-linolenic acid level from zero in the milk supplemented with groundnut oil to 20% in the milk supplemented linseed oil. Eicosapentaenoic acid (EPA) and Docosahexaenoic acid (DHA) level rose from zero in milk with groundnut oil to 3 and 2% respectively in milk supplemented with fish oil (table 3).

TABLE 3
Fatty acid composition milk based product enriched with
omega 3 fatty acids

| | Milk + Ground nut oil | Milk + | Milk + |
|---|---|---|---|
| Fatty acids (mol %) | (control) | Linseed oil | Fish oil |
| Lauric acid | 1.1 | 1.9 | 1.5 |
| Myristic acid | 6.6 | 7.2 | 10.8 |
| Palmitic acid | 28.4 | 24.0 | 32.4 |
| Palmitoleic acid | 1.0 | 1.0 | 4.5 |
| Stearic acid | 6.8 | 10.7 | 9.8 |
| Oleic acid | 39.3 | 23.2 | 23.1 |
| Linoleic acid | 9.3 | 6.5 | 2.5 |
| Linolenic acid | | 20.3 | 1.0 |
| Arachidic acid | 1.8 | | 3.1 |
| Arachidonic acid | | | 2.9 |
| Eicosapentaenoic acid | | | 2.9 |
| Behenic acid | 0.8 | | |
| Docosahexaenoic acid | | | 2.0 |
| Polyunsaturated Fatty | 9.3 | 26.8 | 11.3 |
| Acid (P) | | | |
| Saturated Fatty Acid | 45.5 | 43.9 | 57.6 |
| (S) | | | |
| P/S | 0.20 | 0.61 | 0.20 |

The milk based products enriched with omega 3 fatty acids from natural sources were fed to rats at 16% level of fat for a period 60 days. The total cholesterol in the serum of omega 3 fatty acids enriched milk fed animals was found to be lower by 20-35% compared to those fed on milk based product supplemented with groundnut oil (GNO) which lacks omega 3 fatty acids. Serum triglycerides decreased by 25-29% in omega 3 fatty acid enriched milk based product fed animals compared to GNO supplemented milk-based product (table 4).

It should be further noted that the composition of the present invention is a synergistic composition and is not a mere admixture of the two ingredients because the reduction in the level of cholesterol and triglycerides is much higher for the combination as compared to the individual constituents. This has been demonstrated in Table 4 from where it is clearly concluded that the level of reduction achieved when linseed oil or fish oil is administered is lesser than the level of reduction achieved when the health composition of the present invention comprising milk and the oil is administered. The results of the invention were unexpected and hence, it can not be held obvious to a person skilled in the art.

TABLE 4
Lipid profiles of rats fed milk based product enriched with
groundnut oil, linseed oil or fish oil

| | Total cholesterol | | Triglycerides | |
|---|---|---|---|---|
| | Serum | Liver | Serum | Liver |
| Dietary groups | (mg/dl) | (mg/g) | (mg/dl) | (mg/g) |
| Milk + | 90.2 ± 3.2 | 10.7 ± 0.5 | 136.4 ± 6.4 | 15.1 ± 0.8 |
| Ground nut oil | | | | |
| (control) | | | | |
| Milk + | 62.9 ± 5.7 | 7.5 ± 1.2 | 99.9 ± 12.9 | 12.3 ± 1.0 |
| Linseed oil | | | | |
| Milk + Fish oil | 58.2 ± 6.3 | 8.8 ± 0.6 | 96.4 ± 11.8 | 13.5 ± 0.7 |
| Ground nut oil | 100.3 ± 4.7 | 11.5 ± 1.0 | 151.3 ± 9.1 | 17.1 ± 1.0 |
| Fish oil | 70.4 ± 4.7 | 9.5 ± 0.7 | 111.3 ± 15.5 | 14.0 ± 1.4 |
| Linseed oil | 81.7 ± 3.3 | 8.1 ± 1.2 | 117.4 ± 10.3 | 13.3 ± 2.1 |

The main advantages of the present invention are:

1. The present invention uses fatty acids from natural sources to produce omega 3 fatty acid supplemented milk based product.

2. The process could be employed to obtain commonly consumed milk in the form of reconstitutable powder supplemented with fatty acids of interest.

3. The process can be carried out at pilot plant scale to produce milk based product with omega 3 fatty acid.

4. Nutritionally the product proved to be more beneficial as they contain both medium chain fatty acids from milk and omega 3 fatty acids from natural sources.

5. The product also proved to be advantageous than its individual components in expressing the synergistic hypocholesterolemic and hypotriglyceridemic effect.

1. A synergistic health composition for lowering levels of cholesterol and triglycerides comprising milk or milk-based products and a desired amount of one or more omega 3 fatty acids.

2. A synergistic composition as claimed in claim 1, wherein the ratio between omega 3 fatty acids and milk or milk-based product is in the range of 1:100 to 1:25.

3. A synergistic composition as claimed in claim 1, wherein the ratio between omega 3 fatty acids and milk or milk-based product is 1:50.

4. A synergistic composition as claimed in claim 1, wherein the omega 3 fatty acids are obtained from natural source.

5. A synergistic composition as claimed in claim 1, wherein the omega 3 fatty acids are seleted from α linolenic acid (LNA), eicosapentaenoic acid (EPA) and docosahexaenoic acid (DHA).

6. A synergistic composition as claimed in claim 5, wherein α linolenic acid is obtained from linseed oil or fish oil.

7. A synergistic composition as claimed in claim 5, wherein EPA and DHA are obtained from fish oil.

8. A synergistic composition as claimed in claim 1, wherein the milk based product includes any food grade additions such as proteins, sugars, fats, fibres, colouring agents, preservatives, herbal material, pharmaceuticals, mineral mix and vitamin mix.

9. A synergistic composition as claimed in claim 1, wherein the milk-based product contains:

| Ingredients | Quantity (g) |
|---|---|
| Maltodextrin | 3000 to 3500 |
| Potassium citrate | 50 to 55 |
| Sodium citrate | 50 to 55 |
| K₂HPO₄ | 75 to 100 |
| Skimmed milk powder | 700 to 1000 |
| Calcium gluconate | 300 to 400 |
| Vitamin mix | 25 to 40 |
| Mineral mix | 5 to 10 |
| Sugar | 2000 to 5000 |

for every 50 liters of milk.

10. A synergistic composition as claimed in claim 1, wherein the milk-based product contains:

| Ingredients | Quantity (g) |
|---|---|
| Maltodextrin | 3050.0 |
| Potassium citrate | 52.5 |
| Sodium citrate | 52.86 |
| K₂HPO₄ | 80.34 |
| Skimmed milk power | 720.0 |
| Calcium gluconate | 357.14 |
| Vitamin mix | 30.0 |
| Mineral mix | 8.12 |
| Sugar | 2320.0 |

for every 50 litres of milk.

11. A synergistic composition as claimed in claim 9, wherein the vitamin mix includes vitamin A, vitamin D, vitamin K, thiamine, riboflavin, niacin, vitamin B6, folic acid, biotin, pantothenic acid, choline bitartrate, inositol and vitamin C.

12. A synergistic composition as claimed in claim 10, wherein the milk based product includes multiple Vitamins such as in the range of 200,000 IU vitamin A, 72,000 IU vitamin D, 0.80 g vitamin D, 2.57 mg vitamin K, 51.4 mg thiamine, 128.57 mg riboflavin, 0.771 g niacin, 64.28 mg vitamin B6, 6.42 mg folic acid, 3.57 mg biotin, 257.14 mg pantothenic acid, 2.57 g choline bitartrate, 0.84 g inositol, 7.71 g vitamin C.

13. A synergistic composition as claimed in claim 9, wherein the mineral mix includes FeSO₄. ZnSO₄, CuSO₄, KI and MnSO₄.

14. A synergistic composition as claimed in claim 10, wherein the milk-based product includes 6.42 g FeSO₄ (IP grade), 1.71 g ZnSO₄, 0.12 g CuSO₄, 4.2 mg KI, 10.71 MnSO₄.

15. A synergistic composition as claimed in claim 1, wherein the cholesterol lowering capacity of the composition is in the range of 20-30%.

16. A synergistic composition as claimed in claim 15, wherein the composition lowers the cholesterol levels in serum and liver.

17. A synergistic composition as claimed in claim 1 wherein the triglyceride lowering capacity of the composition is in the range of 20 to 35%.

18. A synergistic composition as claimed in claim 17, wherein the composition lowers the triglyceride levels in serum and liver

19. A process for preparing a synergistic health composition capable of reducing cholesterol and triglyceride levels, said process comprising mixing milk or milk based products with a desired quantum of omega 3 fatty acids.

20. A process as claimed in claim 19, wherein omega 3 fatty acids and milk or milk-based product are mixed in a ratio ranging between 1:100 to 1:25.

21. A process as claimed in claim 20, wherein omega 3 fatty acids and milk or milk-based product are mixed in the ratio of 1:50.

22. A process as claimed in claim 19, wherein the omega 3 fatty acids are obtained from natural sources.

23. A process as claimed in claim 19, wherein the omega 3 fatty acids are selected from α linolenic acid (LNA), eicosapentaenoic acid (EPA) and docosahexaenoic acid (DHA).

24. A process as claimed in claim 23, wherein α linolenic acid is obtained from linseed oil or fish oil.

25. A process as claimed in claim 23, wherein EPA and DHA are obtained from fish oil.

26. A process as claimed in claim 19, wherein the milk-based product includes food grade additions such as proteins, sugars, fats, fibres, colouring agents, preservatives, herbal material, pharmaceuticals, mineral mix and vitamin mix.

27. A process as claimed in claim 19, wherein the milk-based product contains:

| Ingredients | Quantity (g) |
|---|---|
| Maltodextrin | 3000 to 3500 |
| Potassium citrate | 50 to 55 |
| Sodium citrate | 50 to 55 |
| K₂HPO₄ | 75 to 100 |
| Skimmed milk powder | 700 to 1000 |
| Calcium gluconate | 300 to 400 |
| Vitamin mix | 25 to 40 |
| Mineral mix | 5 to 10 |
| Sugar | 2000 to 5000 |

for every 50 liters of milk.

28. A process as claimed in claim 19, wherein the milk-based product contains:

| Ingredients | Quantity (g) |
|---|---|
| Maltodextrin | 3050.0 |
| Potassium citrate | 52.5 |
| Sodium citrate | 52.86 |
| K₂HPO₄ | 80.34 |
| Skimmed milk powder | 720.0 |
| Calcium gluconate | 357.14 |
| Vitamin mix | 30.0 |
| Mineral mix | 8.12 |
| Sugar | 2320.0 |

for every 50 litres of milk.

29. A process as claimed in claim 27, wherein the vitamin mix includes vitamin A, vitamin D, vitamin K, thiamine, riboflavin, niacin, vitamin B6, folic acid, biotin, pantothenic acid, choline bitartrate, inositol and vitamin C.

30. A process as claimed in claim 28, wherein the milk based product includes multiple Vitamins such as in the range of 200,000 IU vitamin A, 72,000 IU vitamin D, 0.80 g vitamin D, 2.57 mg vitamin K, 51.4 mg thiamine, 128.57 mg riboflavin, 0.771 g niacin, 64.28 mg vitamin B6, 6.42 mg folic acid, 3.57 mg biotin, 257.14 mg pantothenic acid, 2.57 g choline bitartrate, 0.84 g inositol, 7.71 g vitamin C.

31. A process as claimed in claim 27, wherein the mineral mix includes FeSO₄, ZnSO₄, CuSO₄, KI and MnSO₄.

32. A process as claimed in claim 28, wherein the milk-based product includes 6.42 g FeSO₄ (IP grade), 1.71 g ZnSO₄, 0.12 g CuSO₄, 4.2 mg KI, 10.71 MnSO₄.

33. A process as claimed in claim 19, wherein said process comprises the steps of:
(a) physically blending milk or milk-based product with omega 3 fatty acids obtained from natural sources to obtain a milk or milk-based product enriched with omega 3 fatty acids;
(b) homogenizing the milk or milk-based product enriched with omega 3 fatty acids of step (a);
(c) partially condensing the homogenized milk or milk-based product of step (b) under vacuum to obtain desired brix, and
(d) spray drying the partially condensed milk or milk based product of step (c) to form powder.

34. A process as claimed in claim 19, wherein the cholesterol lowering capacity of the composition is in the range of 20-30%.

35. A process as claimed in claim 34, wherein the composition lowers the cholesterol level in serum and liver.

36. A process as claimed in claim 19, wherein the triglyceride lowering capacity of the composition is in the range of 20 to 35%.

37. A process as claimed in claim 36, wherein the composition lowers the triglyceride levels in serum and liver.

38. A method for reducing total cholesterol and triglycerides in a subject, said method comprising the step of administering an effective amount of a composition comprising milk or milk-based products and a desired amount of one or more omega 3 fatty acids.

39. A method as claimed in claim 38, wherein the ratio between omega 3 fatty acids and milk or milk-based product is in the range of 1:100 to 1:25.

40. A method as claimed in claim 38, wherein the ratio between omega 3 fatty acids and milk or milk-based product is 1:50.

41. A method as claimed in claim 38, wherein the omega 3 fatty acids are obtained from natural source.

42. A method as claimed in claim 38, wherein the omega 3 fatty acids are seleted from α linolenic acid (LNA), eicosapentaenoic acid (EPA) and docosahexaenoic acid (DHA).

43. A method as claimed in claim 38, wherein α linolenic acid is obtained from linseed oil or fish oil.

44. A method as claimed in claim 38, wherein EPA and DHA are obtained from fish oil.

45. A method as claimed in claim 38, wherein the milk based product includes any food grade additions such as proteins, sugars, fats, fibres, colouring agents, preservatives, herbal material, pharmaceuticals, mineral mix and vitamin mix.

46. A method as claimed in claim 38, wherein the milk-based product contains:

| Ingredients | Quantity (g) |
|---|---|
| Maltodextrin | 3000 to 3500 |
| Potassium citrate | 50 to 55 |
| Sodium citrate | 50 to 55 |
| K₂HPO₄ | 75 to 100 |
| Skimmed milk powder | 700 to 1000 |
| Calcium gluconate | 300 to 400 |
| Vitamin mix | 25 to 40 |
| Mineral mix | 5 to 10 |
| Sugar | 2000 to 5000 |

for every 50 liters of milk.

47. A method as claimed in claim 38, wherein the milk-based product contains:

| Ingredients | Quantity (g) |
|---|---|
| Maltodextrin | 3050.0 |
| Potassium citrate | 52.5 |
| Sodium citrate | 52.86 |
| K₂HPO₄ | 80.34 |
| Skimmed milk powder | 720.0 |
| Calcium gluconate | 357.14 |
| Vitamin mix | 30.0 |
| Mineral mix | 8.12 |
| Sugar | 2320.0 |

for every 50 litres of milk.

48. A method as claimed in claim 46, wherein the vitamin mix includes vitamin A, vitamin D, vitamin K, thiamine, riboflavin, niacin, vitamin B6, folic acid, biotin, pantothenic acid, choline bitartrate, inositol and vitamin C.

49. A method as claimed in claim 47, wherein the milk based product includes multiple Vitamins such as in the range of 200,000 IU vitamin A, 72,000 IU vitamin D, 0.80 g vitamin D, 2.57 mg vitamin K, 51.4 mg thiamine, 128.57 mg riboflavin, 0.771 g niacin, 64.28 mg vitamin B6, 6.42 mg folic acid, 3.57 mg biotin, 257.14 mg pantothenic acid, 2.57 g choline bitartrate, 0.84 g inositol, 7.71 g vitamin C.

50. A method as claimed in claim 46, wherein the mineral mix includes FeSO₄, ZnSO₄, CuSO₄, KI and MnSO₄.

51. A method as claimed in claim 47, wherein the milk-based product includes 6.42 g FeSO₄ (IP grade), 1.71 g ZnSO₄, 0.12 g CuSO₄, 4.2 mg KI, 10.71 MnSO₄.

52. A method as claimed in claim 38, wherein the composition is administered at 10 to 20% level of fat to the subject.

53. A method as claimed in claim 38, wherein the composition is administered for a period of 45 to 90 days.

54. A method as claimed in claim 38, wherein the subject is a mammal including human being.

55. A method as claimed in claim 38, wherein the composition lowers the cholesterol content in the range of 20-30%.

56. A method as claimed in claim 55, wherein the composition lowers the cholesterol level in serum and liver.

57. A method as claimed in claim 38, wherein the composition lowers the triglycerides content by 20 to 35%.

58. A method as claimed in claim 57, wherein the composition lowers the triglyceride levels in serum and liver.
